(19) United States
(12) Patent Application Publication
(10) Pub. No.: US 2025/0231162 A1
(43) Pub. Date: Jul. 17, 2025

(54) GAS MEASURING DEVICE AND METHOD FOR DISPLAYING INFORMATION DETERMINED BY THE GAS MEASURING DEVICE

(71) Applicant: Dräger Safety AG & Co. KGaA, Lübeck (DE)

(72) Inventors: Christoph Clausen, Lübeck (DE); Folke Will, Lübeck (DE)

(21) Appl. No.: 19/020,602

(22) Filed: Jan. 14, 2025

(30) Foreign Application Priority Data
Jan. 15, 2024 (DE) 102024100985.3

(51) Int. Cl. G01N 33/00

(52) U.S. Cl. CPC G01N 33/0063; G01N 33/0068; G01N 33/007; G01N 33/0073

(57) ABSTRACT

The disclosure relates to a gas measuring device having a gas sensor, a first display unit and a control unit. The control unit is configured to determine a hazardous situation based on a gas measurement value of the gas sensor. The first display unit can be controlled to display information related to the hazardous situation. The gas measuring device is characterized in that a second display unit can be controlled to display status information about an internal device status, wherein the control unit is configured to determine the status information. Furthermore, the disclosure relates to a method for displaying information determined by a gas measuring device, wherein the gas measuring device has a first display unit and a second display unit.

### CROSS-REFERENCE TO RELATED APPLICATIONS

This application claims the priority of German Patent Application No. 102024100985.3, filed on Jan. 15, 2024, the entire contents of which being fully incorporated herein by reference.

### DESCRIPTION

The present disclosure relates to a gas measuring device having a first and a second display unit and to a method for displaying information determined by the gas measuring device.

Gas measuring devices are suitable for detecting one or more gases and vapors in the environment and for determining a measured value, as well as for providing information about a hazardous situation using the measured value or values. The hazardous situation indicates whether a dangerous situation, for example a dangerous gas concentration, exists or not. In the case of a dangerous situation, for example, the type of danger is indicated—for example, danger of poisoning; and in the case where no hazardous situation exists, for example, only the current gas measurement values are displayed. To indicate the hazardous situation, the gas measuring device comprises at least one gas sensor that reacts to one or more gases or vapors and generates a measured value. The gas sensors can have different measuring principles, using electrochemical or catalytic gas sensors or infrared gas sensors. Furthermore, the gas measuring device usually comprises a display unit, such as an LED, a horn and/or a display.

Gas measuring devices are available in different designs with different properties and different gas sensor configurations. To distinguish between the different versions, the gas measuring device usually includes a marking that provides, for example, the following information: a type designation, the type of gas sensors installed and/or the gases that can be measured, standards and/or guidelines that the gas measuring device complies with and/or characteristic properties of the gas measuring device, such as a permitted temperature range and a protection class. Such information provides the user of the gas measuring device with important information as to whether the gas measuring device is suitable for his or her purpose and under which conditions the gas measuring device should be operated. The label should be clearly visible and ideally visible at all times so that the user can easily see it and pay attention to the information accordingly.

In the prior art, one or more stickers are usually used to mark a gas measuring device. The stickers are manufactured or printed with the relevant information and attached to the gas measuring device during the production process. Furthermore, it is common for the user to provide an additional marking of the gas measuring device—in particular with regard to the gas sensors with which the gas measuring device is equipped, since the user can change these himself and thus identify which gas sensors are currently included in the gas measuring device and/or which gases can be measured with the current gas measuring device. Stickers are also often used which have a label or a color code—for example, which indicate the installed gas sensors or measurable gases. A major disadvantage of these solutions is that the information cannot be changed or can only be changed with great effort and only manually. If, for example, the gas sensor configuration and/or the list of standards and/or guidelines complied with by the gas measuring device changes, the stickers must be removed and new stickers with the current information must be attached. This results in costs and high time expenditure. Furthermore, it may happen that the information is incorrectly changed or not changed at all. In such cases, it is not possible, or is difficult, for the user to determine from the gas measuring device itself whether the gas measuring device is suitable for a particular application. This may result in the operation having to be aborted or repeated with a suitable gas measuring device.

Based on the solutions known from the prior art and the disadvantages described above, the object of the disclosure is to create a gas measuring device which efficiently provides the user with current information relating to the gas measuring device, wherein the information relates to an internal device status. Changes to the information should be able to be made available as quickly as possible.

Features and details which are described in connection with the gas measuring device according to the disclosure also apply in connection with the method according to the disclosure, so that with regard to the disclosure of the individual aspects of the disclosure, reference is always made or rather can be made to each other.

The gas measuring device according to the disclosure comprises a gas sensor, a first display unit and a control unit. The gas measuring device can comprise a single gas sensor or a plurality of gas sensors. The control unit is designed to determine a hazardous situation based on a gas measurement value from the gas sensor. Furthermore, the first display unit can be controlled by the control unit in such a way that information relating to the hazardous situation can be displayed. According to the disclosure, the gas measuring device is characterized in that a second display unit is provided and can be controlled in such a way that status information about at least one internal device status can be displayed, wherein the control unit is configured to determine this status information.

The gas measuring device according to the disclosure can comprise one or more gas sensors. A gas sensor is a sensor for detecting one or more gases or vapors. The gas sensors can have different measuring principles and can be designed, for example, as electrochemical or catalytic gas sensors, infrared sensors or photoionization detectors (PID).

A control unit is a unit that is suitable for determining and/or processing data, such as gas measurement values and internal device statuses, and for generating a control signal, for example for a display unit. The control unit can have one or more computing units, wherein a computing unit comprises, for example, a processor, a microprocessor or a field programmable gate array (FPGA). Multiple computing units can be connected to each other via an interface for data exchange. Furthermore, it is conceivable that the computing units are spatially separated from one another, for example, mounted on different circuit boards.

The gas measuring device is able to generate a gas measurement value using the gas sensor. The control unit is designed to determine a hazardous situation based on the gas measurement value. A hazardous situation exists, for example, in the case of a gas alarm if the gas measurement value is greater than or less than a threshold value. Furthermore, the control unit is configured to control the first display unit such that the first display unit displays information relating to the hazardous situation. The first display unit comprises components for acoustic and/or haptic output and/or optical display, for example a horn, a vibration motor, an LED (light-emitting diode) and/or a display. Preferably, the first display unit comprises at least one LED. Particularly preferably, the first display unit comprises a display, a horn and at least one LED.

The type of information output relating to the hazardous situation indicated by the first display unit depends on the given component of the first display unit. In the case of a gas alarm situation occurring, for example, the display indicates the gas alarm, indicating what type of gas alarm it is and/or how the gas measurement value deviates from the threshold value. Furthermore, for example, the horn, the vibration motor and/or the at least one LED are active at least once so that their optical, haptic and/or acoustic signal is perceptible to the user.

In the case of a non-existent gas alarm, i.e. in the case where the determined hazardous situation indicates that there is no dangerous situation, the display only shows, for example, the current gas measurement value as information related to the hazardous situation. Furthermore, for example, the horn, the vibration motor and/or at least one LED are inactive in this case. It is conceivable that the horn, the vibration motor and/or at least one LED are also active at least once in this case. However, the type of information output, i.e. the activation of the horn, the vibration motor and/or at least one LED, is different from the previously described activation in the case of a gas alarm situation occurring. In any case, the information output relating to the hazardous situation is designed in such a way that it can be distinguished by the user according to the determined hazardous situation.

The control unit is additionally configured to determine status information about an internal device status and to control the second display unit such that the second display unit displays this status information. The internal device status includes information and/or current properties relating to the hardware and/or software of the gas measuring device, such as a configuration, a sensor configuration, an error status and/or a compliance with standards. The control unit determines the status information at least partially on the basis of data stored on the gas measuring device. The status information is determined, for example, on the basis of configuration settings of the gas measuring devices, wherein these configuration settings can be changed by the user. Furthermore, the determination of the status information relates to a check of a specific condition, a property of the gas measuring device, in particular a property that can be changed, and/or a comparison of configuration settings. Exemplary embodiments for determining the status information are explained in the further course of the description. Furthermore, it is conceivable that the control unit determines the status information at least partially on the basis of data which the gas measuring device queries from an external communication partner. The external communication partner can, for example, be a control room that provides data related to the gas measuring device.

The second display unit preferably comprises a display for displaying the status information. Particularly preferably, the second display unit is mounted together with the first display unit on or in a housing of the gas measuring device. For example, the first display unit is arranged on a first side of the housing and the second display unit is arranged on a second, opposite side of the housing of the gas measuring device. Furthermore, it is conceivable that the second display unit is attached to another housing of the gas measuring device, wherein the gas measuring device comprises at least two housings which are spatially separated from one another. For example, a gas sensor, a first computing unit and the first display unit are arranged on or in a first housing, and a second computing unit and the second display unit are arranged on or in a second housing. The control unit therefore comprises two computing units that are connected to each other via an interface for data exchange.

The particular advantage of the gas measuring device according to the disclosure is that, in addition to information relating to a gas measurement value, the user receives status information via a display, i.e. information relating to an internal device status, wherein the status information indicates the current internal status of the gas measuring device. The control unit of the gas measuring device determines the status information and controls the second display unit to display the determined status information. This advantageously ensures that the user can always efficiently identify the current internal status of the gas measuring device, with changes in the internal status being apparent to the user because the status information is determined and displayed by the second display unit.

Furthermore, the first and second display units are advantageously independent of each other, so that their embodiments are flexible and adaptable to the given application. For example, the maximum energy consumption, the size and/or the area of application of the gas measuring device can be taken into account.

According to an exemplary embodiment of the gas measuring device, the second display unit is a bistable display unit. Furthermore, the second display unit preferably comprises electronic paper, also referred to as e-paper or epaper. The terms “bistable display unit” and “display unit with electronic paper” refer to a display unit that maintains its state without any voltage applied. Information that was previously displayed will continue to be shown even when the display unit is not powered. Preferably, the bistable display unit is a display whose pixels retain the state when the maintenance voltage or operating voltage is removed.

A gas measuring device with a second display unit in the form of a bistable display unit or a display unit comprising electronic paper has the advantage that the information of the second display unit, i.e. the status information about the internal device status of the gas measuring device, is recognizable to the user at any time. This applies even if the gas measuring device is not in operation, i.e. is switched off or disconnected from a power supply. This means that the user can use the status information to check whether the gas measuring device is suitable for a specific application even if the gas measuring device is switched off or de-energized. Furthermore, the bistable display unit and the display unit having electronic paper offer the advantage of being energy-saving, which is particularly advantageous for rechargeable or battery-operated gas measuring devices, which in this case can be operated for a longer period. A particular advantage of electronic paper is its high-contrast display as well as easy control and low costs compared to a standard display.

In an exemplary embodiment of the gas measuring device, the control unit is configured to detect a gas sensor configuration, a device identification, a device property, a software version, a configuration and/or a compliance with standards and, based thereon, to determine the status information and to display it on the second display unit. The control unit is advantageously designed to determine different internal device statuses.

The gas sensor configuration indicates which gas sensors the gas measuring device includes or which gas sensors are currently configured to measure gas. The control unit is configured to detect which gas sensors the gas measuring device includes or which gas sensors are currently activated for measurement, and uses this to determine status information that indicates which gases can be measured by the gas measuring device or which gases are being measured.

The device identification indicates the type and/or version of a gas measuring device. The device identification may change, for example, after a repair of the gas measuring device, in which a circuit board of the gas measuring device is replaced, or if the gas measuring device is extended by an additional component. An additional component can be, for example, a component for wireless communication or a component with a pump for suctioning ambient air to the sensor. The control unit is configured to detect the device identification, for example by checking the version of the circuit board and using this version to determine the type and/or version of the gas measuring device, wherein the assignment is stored in the gas measuring device, for example in the form of a table. Furthermore, the control unit is configured to detect the device identification, for example by detecting additional components attached to the gas measuring devices and determining the type of gas measuring device, wherein, for example, the possible types of gas measuring device with the respective additional components are stored in tabular form.

The device property indicates the extent to which the gas measuring device is operational, for example a battery status or an error state of the gas measuring device. For example, the control unit is designed to detect the battery status and use this to determine whether or for how long the gas measuring device can be used. Furthermore, the control unit is designed, for example, to detect whether and which errors are currently present and to determine from this whether the gas measuring device is operational despite any errors that may have occurred. The errors mentioned can be hardware-related errors, such as a defective component or a detected fall of the gas measuring device, or software-related errors, such as a failed adjustment of the gas sensors or an expired interval. In an advantageous manner, the user of the gas measuring device can therefore recognize whether the gas measuring device has sufficient energy capacity for a certain period of use or whether the gas measuring device is operational.

The compliance with standards indicates which standards and/or guidelines the gas measuring device can fulfill and/or according to which standard the gas measuring device is designed. The control unit is configured to determine which standards and/or directives are met and/or implemented. The control unit checks, for example, the configuration settings and/or the software version of the gas measuring device. Certain standards and/or guidelines require certain configuration settings of the gas measuring device. The control unit compares the current configuration settings of the gas measuring device with the required configuration settings necessary to meet a standard and/or a directive and determines those standards and/or directives that are currently met by the gas measuring device. Furthermore, the control unit checks, for example, which standards and/or guidelines the current software version of the gas measuring device complies with. In practice, it may happen that different software versions meet different standards and/or guidelines, especially if approval has only been granted for certain software versions or if requirements regarding the standards and/or guidelines change. The control unit therefore detects the current software version and determines the standards and/or guidelines currently fulfilled by the gas measuring device, wherein an assignment of the respective software version to the fulfilled standards and/or guidelines is stored, for example, in tabular form. Advantageously, the user of the gas measuring device can thus check whether the standards and/or directives fulfilled by the gas measuring device correspond to the standards and/or directives required by its application.

Advantageously, the control unit, as described in the previous paragraphs, determines the current status information, which is then displayed on the second display unit, so that the user can see what the current internal device status of the gas measuring device is and can accordingly assess whether the gas measuring device is suitable for a specific application scenario.

According to an exemplary embodiment of the gas measuring device, the control unit is configured to compare a currently determined status information with a previously determined status information. Furthermore, if a difference is detected between the current status information and the previously determined status information, the second display unit can be controlled to display the current status information instead of the previously determined status information. Advantageously, the second display unit is controlled as required, i.e. in the case in which a currently determined status information about the internal device status of the gas measuring device differs from a previously determined status information which is already displayed on the second display unit. This is a particularly efficient way of providing and displaying information. Furthermore, the control unit is preferably configured to determine the status information during a device switch-on process and/or a device switch-off process. This also contributes to an efficient way of providing information.

In an exemplary embodiment of the gas measuring device, the control unit is configured to detect a device configuration change and to determine the status information when the device configuration changes. The device configuration change takes place, for example, through user input on the gas measuring device or through an external configuration application. The device configuration change includes, for example, a change in the setting of the gas measuring device. Advantageously, the determination of the status information takes place immediately adjacent to the point in time at which the probability of a change in the status information is high.

According to an exemplary embodiment of the gas measuring device, the second display unit can be controlled such that the second display unit alternately displays different information. The different information includes in particular status information determined by the control unit. These different pieces of information are divided by the control unit, the amount of each divided piece of information being dependent on the size of the second display unit, and are displayed alternately on the second display unit at an interval, for example of five seconds. The interval should be selected and implemented by the control unit in such a way that it gives the user enough time to absorb all the information that is currently being displayed on the second display unit. In an advantageous way, the information is presented on the second display unit in a clear and easily perceptible manner.

Furthermore, the disclosure relates to a method for displaying information determined by a gas measuring device, wherein the gas measuring device has a first display unit and a second display unit, comprising the following steps:
determining a hazardous situation based on a gas measurement value from a gas sensor of the gas measuring device,
displaying information related to the hazardous situation on the first display unit,
determining status information about an internal device status, and
displaying status information on the second display unit.

Determining the hazardous situation involves, for example, comparing the gas measurement value with a threshold value to determine whether a gas alarm situation is occurring. Based on the hazardous situation, information is displayed on a first display unit. The first display unit is suitable, for example, to output an optical signal, acoustic and/or haptic information, and to inform the user of the gas measuring device about the hazardous situation. The determination of the hazardous situation is carried out by a control unit of the gas measuring device, which, as previously described, can comprise one or more computing units. For example, the determination of the hazardous situation and the display of the information on the first display unit is repeated every second if the gas measuring device is switched on.

In addition to the hazardous situation, the control unit determines the status information. For example, the control unit checks the configuration settings of the gas measuring device for compliance with certain requirements to fulfill a standard and/or a directive and determines status information that indicates which standards and/or directives are currently fulfilled by the gas measuring device. Furthermore, the control unit detects, for example, the active gas sensors and determines status information that indicates which gases are currently being measured. The control unit controls the second display unit so that it displays the status information. The determination of the status information and the display of the status information on the second display unit is repeated, for example, as needed, i.e. when a setting of the gas measuring device is changed and/or when certain events occur, such as the gas measuring device being switched on and off. Preferably, the second display unit is designed such that the status information is also displayed when the gas measuring device is switched off. The second display unit includes, for example, electronic paper, which displays information even when the power is off.

The method according to the disclosure offers the particular advantage that information related to a gas measurement value is determined and displayed, and also status information about the internal device status of the gas measuring device is determined and displayed. Furthermore, the status information is updated so that the user can see whether the gas measuring device is suitable for its intended use in its current state.

Further features, objects and effects of the invention will become apparent from the following description of specific exemplary embodiments and the accompanying figures. Exemplary embodiments of the invention are described without limiting the general inventive concept.

In the figures:

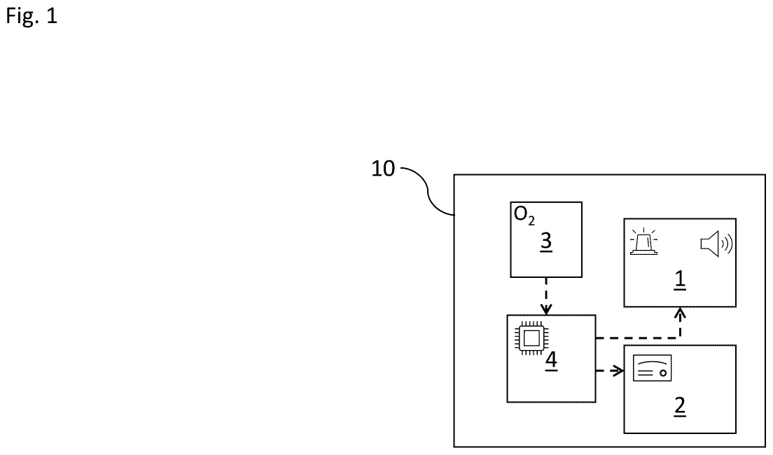

FIG. 1 is a schematic block diagram of an embodiment of the gas measuring device according to the disclosure,

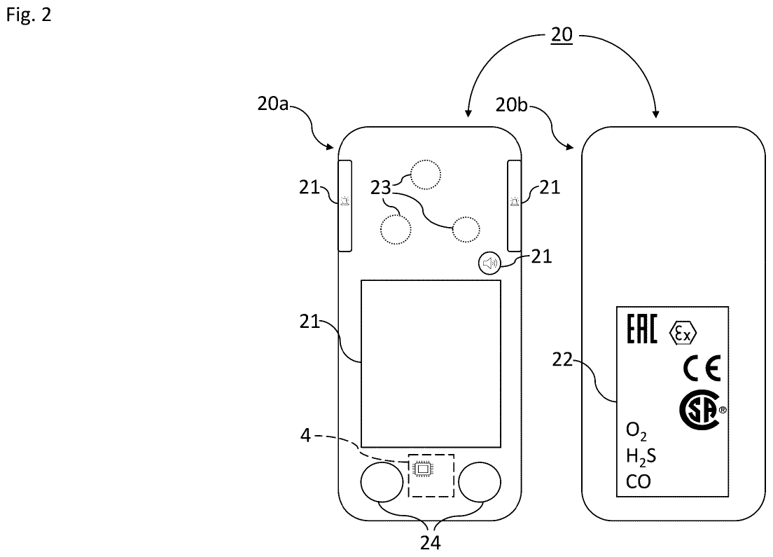

FIG. 2 is an additional schematic representation of an embodiment of the gas measuring device according to the disclosure, and

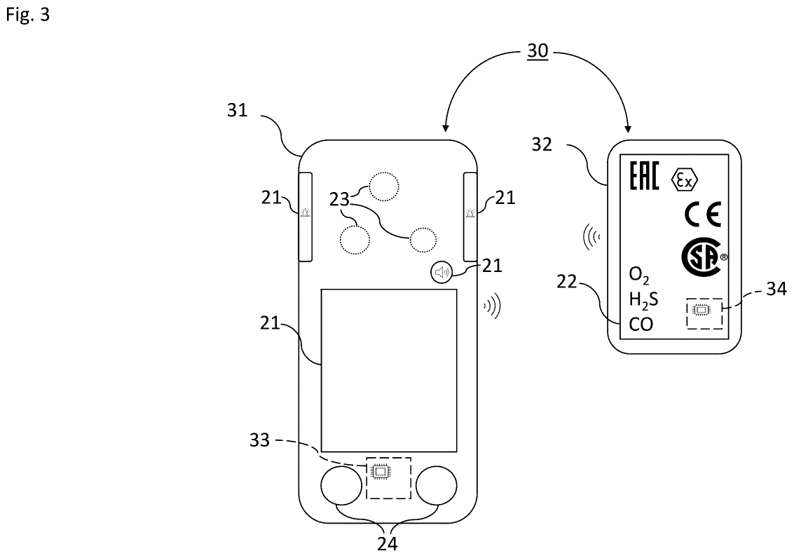

FIG. 3 is a schematic representation of a further embodiment of the gas measuring device according to the disclosure.

Exemplary embodiments of the invention are described in detail below with reference to the accompanying figures. Similar components in multiple figures are each provided with the same reference symbols.

FIG. 1 shows an exemplary embodiment of the gas measuring device 10 according to the disclosure using a schematic block diagram with a control unit 4 which is electronically connected to a first display unit 1 and a second display unit 2. The first display unit 1 comprises a red and a green LED as well as a horn. The second display unit 2 comprises electronic paper, also called e-paper or epaper. Furthermore, the gas measuring device comprises a gas sensor 3, in this case an electrochemical gas sensor for measuring an oxygen concentration, which is electronically connected to the control unit 4. The electronic connections mentioned are shown as dashed lines.

The control unit 4 of the gas measuring device 10 is configured to determine a hazardous situation based on a gas measurement value of the gas sensor 3. The control unit 4 compares the gas measurement value with a threshold value, wherein a gas alarm situation occurs if the threshold value is exceeded. In the event that the gas measurement value is less than the threshold value and a gas alarm situation has occurred, the control unit 4 controls the first display unit 1 in such a way that the red LED and the horn signal the gas alarm. In the event that the gas measurement value is less than the threshold value and there is no gas alarm situation, the control unit 4 controls the first display unit 1 in such a way that the green LED is activated to signal that there is no gas alarm situation. This advantageously allows a user—who is not shown—to see directly whether a gas alarm situation is occurring or not.

The control unit 4 is further configured to determine status information about an internal device status of the gas measuring device 10 and to control the second display unit 2 to display this status information. The control unit 4 is able to determine different status information, in particular gas sensor configuration and/or compliance with standards.

The control unit 4 detects that the gas measuring device comprises an electrochemical gas sensor 3 and that this is currently measuring an oxygen concentration. The control unit 4 detects, for example via a detector for unplugged connections, that the gas sensor 3 is physically plugged in and that the gas sensor 3 generates oxygen gas measurement values.

The control unit controls the second display unit 2 in such a way that this status information is displayed. In this case, the status information is that an oxygen concentration is currently being measured. For example, the molecular formula of oxygen appears on the second display unit 2.

Furthermore, the control unit 4 detects current configuration settings as well as the version of the software installed on the gas measuring device 10, which have an influence on the fulfillment of a standard and/or directive. An assignment of which standards and/or guidelines are fulfilled with which software version and with which configuration settings is stored in the gas measuring device 10. The control unit 4 thus uses the assignment to determine which standards and/or guidelines are currently fulfilled, and controls the second display unit 2 in such a way that it displays the fulfilled standards and/or guidelines in the form of text or symbols.

Advantageously, the gas measuring device 10 determines the current internal device status of the gas measuring device 10 and displays it on the second display unit 2. This enables a user to efficiently evaluate whether the gas measuring device 10 is suitable for his application.

FIG. 2 shows a schematic representation of an exemplary embodiment with two views of the gas measuring device 20 according to the disclosure, wherein the top view of the front side 20a and the top view of the rear side 20b of the gas measuring device are shown. The gas measuring device comprises a first display unit 21 and a second display unit 22, which are mounted in a single housing of the gas measuring device 20. Furthermore, the gas measuring device comprises three gas inlets 23 for three electrochemical gas sensors (not shown) for measuring, for example and as shown in FIG. 2, an oxygen concentration, a hydrogen sulfide concentration and a carbon monoxide concentration, as well as two buttons 24 for operation of the gas measuring device by a user.

The gas measuring device 20 comprises, like the gas measuring device 10 in FIG. 1, a control unit 4. The control unit 4 is designed to determine a hazardous situation on the basis of the gas measurement values of the three gas sensors (not shown) and to control the first display unit 21 in such a way that information relating to the hazardous situation is displayed.

The determination of the hazardous situation by the control unit and the control and display of the first display unit 21 is substantially carried out as set out in the description of FIG. 1. In contrast to the gas measuring device 10 in FIG. 1, the display unit 21 of the gas measuring device 20 in FIG. 2 additionally comprises a display 21, which can also be controlled to display information relating to the hazardous situation. The display 21 is able to indicate which of the three gas sensors has a gas alarm situation and what type of gas alarm it is: warning, pre-alarm or primary alarm.

The determination of the status information by the control unit and the control of the second display unit 22, which also comprises electronic paper, is carried out in accordance with the description of the gas measuring device 10 in FIG. 1. The second display unit 22 is controlled by the control unit in such a way that it displays the molecular formulas of the currently measured gases, for example and as shown in FIG. 2, oxygen, hydrogen sulfide and carbon monoxide, as well as the standards and/or guidelines currently met by the gas measuring device 20, in this case a CE marking, a CSA marking and an EAC Ex marking for explosion-proof devices.

FIG. 3 shows a schematic representation of an example embodiment of the gas measuring device 30 according to the disclosure, wherein the gas measuring device comprises a first housing 31 with the first display unit 21, the three gas sensor inlets 23 and the three gas sensors (not shown) as well as the two buttons 24 and a second housing 32 with the second display unit 22. The gas measuring device 30 substantially corresponds to the gas measuring device 20 in FIG. 2—with regard to the same components reference is made to the description of FIG. 2. In contrast to the gas measuring device 20 in FIG. 2, the control unit comprises a first computing unit 33 and a second computing unit 34, wherein the first housing 31 comprises the first computing unit 33 and the second housing 32 comprises the second computing unit 34. The two computing units 33, 34 are wirelessly connected to each other via an interface for data exchange. According to the description of FIG. 2, the control unit determines the hazardous situation with the first computing unit 33 and controls the first display unit 21 in such a way that information related to the hazardous situation is displayed. Also in accordance with the description of FIG. 2, the control unit with the second computing unit 34 determines the status information about the internal device status of the gas measuring device 30 and controls the second display unit in such a way that this status information is displayed.

Advantageously, the first display unit 21 and the second display unit 22 of the gas measuring device 30 in FIG. 3 are spatially separated from one another, so that a user of the gas measuring device 30 is able to position the gas measuring device 30 according to the respective application such that the status information and the information relating to a hazardous situation is easily perceptible as appropriate to the application.

### LIST OF REFERENCE SIGNS

1 first embodiment of a first display unit
2 first embodiment of a second display unit
3 gas sensor
4 control unit
10 first embodiment of a gas measuring device
20 second embodiment of a gas measuring device
20a front of the gas measuring device
20b back of the gas measuring device
21 second embodiment of a first display unit
22 second embodiment of a second display unit
23 gas sensor inlets
24 buttons
30 third embodiment of a gas measuring device
31 first housing of the gas measuring device
32 second housing of the gas measuring device
33 control unit with a first computing unit
34 control unit with a second computing unit

1. A gas measuring device comprising:
a gas sensor;
a first display unit;
wherein the first display unit is configured to display information relating to a hazardous situation:
a control unit;
wherein the control unit is configured to determine the hazardous situation on the basis of a gas measurement value of the gas sensor;
wherein the control unit is configured to determine a status information about an internal device status; and
a second display unit:
wherein the second display unit is configured to display the status information;
wherein the second display unit is a bistable display unit.

2. The gas measuring device according to claim 1, wherein the second display unit comprises electronic paper.

3. The gas measuring device of claim 1, wherein the control unit is configured to detect a gas sensor configuration, a device identification, a device property, a software version, and compliance with standards, and to determine the status information based thereon.

4. The gas measuring device of claim 1, wherein the control unit is configured to compare the status information with a previously determined status information and, if a difference is determined between the status information and the previously determined status information, the second display unit displays the current status information.

5. The Gas measuring device of claim 1, wherein the control unit is configured to determine the status information during a device switch-on process and a device switch-off process.

6. The gas measuring device of claim 1, wherein the control unit is configured to detect a device configuration change and to determine the status information in the event of a change in the device configuration.

7. The gas measuring device of claim 1, wherein the second display unit is configured to displays different information alternately at an interval.

8. A method for displaying information determined by a gas measuring device comprising:
determining a hazardous situation based on a gas measurement value of a gas sensor of the gas measuring device;
displaying information related to the hazardous situation on a first display unit;
determining status information about an internal device status; and
displaying the status information on a second display unit.

9. The method according to claim 8, wherein the second display unit comprises electronic paper.

10. The method according to claim 8, further comprising detecting a gas sensor configuration, a device identification, a device property, a software version, and compliance with standards and determining the status information based thereon.

11. The method according to claim 8, further comprising comparing the status information with a previously determined status information and, if a difference is determined between the status information and the previously determined status information, displaying a current status information.

12. The method according to claim 8, further comprising determining the status information during a device switch-on process and a device switch-off process.

13. The method according to claim 8, further comprising detecting a device configuration change and determining the status information in the event of a change in the device configuration.

14. The method according to claim 8, further comprising displaying different information alternately at an interval.
